(19) United States
(12) Patent Application Publication
(10) Pub. No.: US 2012/0129209 A1
(43) Pub. Date: May 24, 2012

(54) ALIGNING CELLS ON WRINKLED SURFACE

(75) Inventors: Michelle Khine, Irvine, CA (US); Jesus Isaac Luna, Merced, CA (US)

(73) Assignee: THE REGENTS OF THE UNIVERSITY OF CALIFORNIA

(21) Appl. No.: 13/257,019

(22) PCT Filed: Mar. 18, 2010
(86) PCT No.: PCT/US10/27849

§ 371 Date: Feb. 14, 2012

Related U.S. Application Data
(60) Provisional application No. 61/161,738, filed on Mar. 19, 2009.
(60) Provisional application No. 61/177,402, filed on May 12, 2009.

(51) Int. Cl. C12N 5/071; C12N 5/079; C12Q 1/02; C12N 5/077

(52) U.S. Cl. 435/29; 435/402; 435/363; 435/354; 435/366

(57) ABSTRACT

A method is provided for preparing an aligned cell population comprising the culturing one or more cells on a surface having a texture, which texture has an average height of from about 100 nanometers to about 5 micrometers, thereby forming an aligned cell population on the textured surface. Also provided is a method to prepare the surface which method comprises the steps of: a) depositing a metal onto an unstressed or pre-stressed thermoplastic material; b) reducing the surface area of the receptive material by at least about 60%; and c) preparing the surface via lithography.

### CROSS-REFERENCE TO RELATED APPLICATIONS

This application claims the benefit under 35 U.S.C. §119(e) of U.S. Provisional Application Ser. Nos. 61/161,738, filed Mar. 19, 2009 and 61/177,402, filed May 12, 2009, the content of each of which is hereby incorporated by reference into the present disclosure.

### BACKGROUND OF THE INVENTION

Throughout this disclosure, various technical and patent publications are referenced to more fully describe the state of the art to which this invention pertains. These publications are incorporated by reference, in their entirety, into this application.

Tissue engineering is the use of a combination of cells, engineering and materials methods, and suitable biochemical and physio-chemical factors to improve or replace biological functions. Engineered tissues can be used to repair or replace portions of or whole tissues (i.e., bone, cartilage, blood vessels, bladder, etc.).

Often, the tissues involved require certain mechanical and structural properties for proper functioning. Efforts have been made to make tissues that perform specific biochemical functions using cells within an artificially-created support system (e.g. an artificial pancreas, or a bioartificial liver).

Examples of engineered tissues include bioartificial liver device, artificial pancreas, artificial bladders, cartilage, Doris Taylor's heart in a jar, tissue-engineered airway, artificial skin constructed from human skin cells embedded in collagen, and artificial bone marrow.

Advances in stem cell biology made it possible for myocardial regeneration after infarction. Myocardium may be formed in infarcted rodent hearts using human embryonic stem cell (hESC)—derived cardiomyocytes. However, small and highly variable cell to graft size limits its application. In some methods, dispersed cells are injected enzymatically or mechanically directly into the injured left ventricular wall. Advances in cardiac tissue engineering, which seek to generate myocardium-like tissue in vitro and then implant the tissue in vivo, provide better ways to control cell seeding efficiency and graft size.

In tissue engineering, cells are often implanted or ‘seeded’ into an artificial structure capable of supporting three-dimensional tissue formation. These structures, typically called scaffolds, are often critical, both ex vivo as well as in vivo, to recapitulating the in vivo milieu and allowing cells to influence their own microenvironments. Scaffolds usually serve at least one of the following purposes: allow cell attachment and migration; deliver and retain cells and biochemical factors; enable diffusion of vital cell nutrients and expressed products; and exert certain mechanical and biological influences to modify the behavior of the cell phase.

Substrate topographical features of the scaffolds have a great impact on cell attachment and migration and morphology. Surface roughness can affect biocompatibility. Optimal micro-roughness depends on cell type. Nano scale roughness is reported to promote cell attachment, migration and proliferation. This may be partly due to a similarity between the nano scale roughness of the scaffold surface and nanostructures found in natural extra-cellular matrix such as nano-stripes in collagen.

A number of technologies have been introduced to make rough surface structures, including nano imprint lithography (Charest et al. (2005) J Vac Sci Technol B 23:3011-4), laser holography (Clark et al. (2002) Int J Biochem Cell Biol 34:816e25; Clark et al. (1991) J Cell Sci 99:73-7), nano-lithography (Arnold et al. (2004) ChemPhysChem 5:383-8), nano imprinting methods (Lenherta et al. (2005) Biomaterials 26:563-70), laser machining (Lu et al. (2003) Mater Lett 58:29-32; Mirzadeh (2003) Radiat Phys Chem 67:381-5; Zhu et al. (2004) J Biomed Mater Res B 70B:43-8; Wang et al. (2006) J Biomed Mater Res A 78:746-54), electrospinning (Schindler et al. (2005) Biomaterials 26:5624-31) and polymer demixing (Dalby et al. (2004) Cell Biol Int 28:229-36). However, these methods require costly manufacture of devices. There is a need to develop a cost effective method to make rough surface structures suitable for tissue engineering.

### SUMMARY OF THE INVENTION

This invention provides a new method to align or grow cells on a textured, or alternatively termed “wrinkle”, surface. The invention also provides methods to make the textured surface conveniently at a very low cost. The invention is useful for tissue engineering, such as generating a cardiac patch.

Thus in one aspect, this invention provides a method for preparing an aligned cell population comprising the steps of: 1) placing one or more cells on a surface having a texture, which texture has an average height of from about 100 nanometers to about 5 micrometers; and 2) allowing the cells to migrate or divide on the surface, thereby forming an aligned cell population on the textured surface. The method also comprises culturing one or more cells on a surface having a texture, which texture has an average height of from about 100 nanometers to about 5 micrometers, thereby forming an aligned cell population on the textured surface.

In one aspect, preparation of the textured surface comprises the steps of: a) depositing a metal onto an unstressed or pre-stressed receptive thermoplastic material; b) reducing the surface area of the receptive thermoplastic material by at least about 60%; and c) preparing the surface via lithography.

In some embodiments, the cell placed on the textured surface is an isolated stem cell. In one aspect, the isolated stem cell is an embryonic stem cell, a pluriopotent stem cell, a somatic stem cell and or iPS stem cell. In another aspect, the cell is a fetal or neonatal cardiac cell.

### BRIEF DESCRIPTION OF THE DRAWINGS

FIG. 1 shows alignment of cardiac cells. 1A shows that cardiac cells placed on a flat surface of a polydimethylsiloxane (PDMS) base did not align after 24 hours culturing. 1B shows that cardiac cells placed on a textured surface of a PDMS base, as prepared by methods of this invention, align into clusters of cells.

FIG. 2 shows cell alignment after different period of time on surfaces without texture or with different sizes of texture.

### DETAILED DESCRIPTION OF THE INVENTION

As used herein, certain terms may have the following defined meanings.

As used in the specification and claims, the singular form “a,” “an” and “the” include plural references unless the context clearly dictates otherwise. For example, the term “a microfluidic channel” includes a plurality of microfluidic channels.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps for preparing the microfluidic device. Embodiments defined by each of these transition terms are within the scope of this invention.

A “thermoplastic material” is intended to mean a plastic material which shrinks upon heating. In one aspect, the thermoplastic materials are those which shrink uniformly without distortion. A “Shrinky-Dink” is a commercial thermoplastic which is used a children's toy. The shrinking can be either bi-axially (isotropic) or uni-axial (anisotropic). Suitable thermoplastic materials for inclusion in the methods of this invention include, for example, high molecular weight polymers such as acrylonitrile butadiene styrene (ABS), acrylic, celluloid, cellulose acetate, ethylene-vinyl acetate (EVA), ethylene vinyl alcohol (EVAL), fluoroplastics (PTFEs, including FEP, PFA, CTFE, ECTFE, ETFE), ionomers kydex, a trademarked acrylic/PVC alloy, liquid crystal polymer (LCP), polyacetal (POM or Acetal), polyacrylates (Acrylic), polyacrylonitrile (PAN or Acrylonitrile), polyamide (PA or Nylon), polyamide-imide (PAI), polyaryletherketone (PAEK or Ketone), polybutadiene (PBD), polybutylene (PB), polybutylene terephthalate (PBT), polyethylene terephthalate (PET), Polycyclohexylene Dimethylene Terephthalate (PCT), polycarbonate (PC), polyhydroxyalkanoates (PHAs), polyketone (PK), polyester polyethylene (PE), polyetheretherketone (PEEK), polyetherimide (PEI), polyethersulfone (PES), polysulfone polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC), and spectralon. A “Shrinky-Dink” is a commercial thermoplastic material which is marketed as a children's toy. As used herein, the terms “thermoplastic base” and “thermoplastic cover” refer to thermoplastic material having been subjected to both the etching process as well as heating process. The “thermoplastic base” would be located at the bottom or within the device, and the “thermoplastic cover” is the last layer of one or more layers of thermoplastic base.

A “solution” is intended to refer to a substantially homogeneous mixture of a solute, such as a solid, liquid, or gaseous substance, with a solvent, which is typically a liquid. The solution can be either aqueous or non-aqueous. Examples of suitable solutes in solutions include fluorescent dyes, biological compounds, such as proteins, DNA and plasma, and soluble chemical compounds. Examples of suitable solids include beads, such as polystyrene beads, and powders, such as a metal powder. A “suspension” is intended to refer to a substantially heterogeneous fluid containing a solid, wherein the solid is dispersed throughout the liquid, but does not substantially dissolve. The solid particles in a suspension will typically settle as the particle size is large, compared to a colloid, where the particle size is small such that the suspension does not settle. Examples of suitable suspensions include biological suspensions such as whole blood, cell compositions, or other cell containing mixtures. It is contemplated that any solution, solid or suspension can be mixed using the mixers disclosed herein, provided that the solid has a particle size sufficiently small to move throughout the channels in the mixer.

In general, the image-forming material is one which is compressed upon heating, bonds to the plastic and is durable (can be used as a mold for multiple iterations). For example, “image-forming material” is, in one aspect, intended to mean a composition, typically a liquid, containing various pigments and/or dyes used for coloring a surface to produce an image or text such as ink and printer toner. In addition to an ink, the image forming material can be a metal, such as gold, titanium, silver, a protein, a colloid, a dielectric substance, a paste or any other suitable metal or combination thereof. Examples of suitable proteins include biotin, fibronectin and collagen. Examples of suitable colloids include pigmented ink, paints and other systems involving small particles of one substance suspended in another. Examples of suitable dielectric substances include metal oxides, such as aluminum oxide, titanium dioxide and silicon dioxide. Examples of suitable pastes include conductive pastes such as silver pastes.

The image forming material can be applied to the thermoplastic material by a variety of methods known to one skilled in the art, such as printing, sputtering and evaporating. The term “evaporating” is intended to mean thermal evaporation, which is a physical vapor deposition method to deposit a thin film of metal on the surface of a substrate. By heating a metal in a vacuum chamber to a hot enough temperature, the vapor pressure of the metal becomes significant and the metal evaporated. It recondenses on the target substrate. As used herein, the term “sputtering” is intended to mean a physical vapor deposition method where atoms in the target material are ejected into the gas phase by high-energy ions and then land on the substrate to create the thin film of metal. Such methods are well known in the art (Bowden et al. (1998) Nature (London) 393: 146-149; Bowden et al. (1999) Appl. Phys. Lett. 75: 2557-2559; Yoo et al. (2002) Adv. Mater. 14: 1383-1387; Huck et al. (2000) Langmuir 16: 3497-3501; Watanabe et al. (2004) J. Polym. Sci. Part B: Polym. Phys. 42: 2460-2466; Volynskii et al. (2000) J. Mater. Sci. 35: 547-554; Stafford et al. (2004) Nature Mater. 3: 545-550; Watanabe et al. (2005) J. Polym. Sci. Part B: Polym. Phys. 43: 1532-1537; Lacour, et al. (2003) Appl. Phys. Lett. 82: 2404-2406.)

In addition, the image forming material can be applied to the thermoplastic material using “pattern transfer”. The term “pattern transfer” refers to the process of contacting an image-forming device, such as a mold or stamp, containing the desired pattern with an image-forming material to the thermoplastic material. After releasing the mold, the pattern is transferred to the thermoplastic material. In general, high aspect ratio pattern and sub-nanometer patterns have been demonstrated. Such methods are well known in the art (Sakurai, et al., U.S. Pat. No. 7,412,926; Peterman, et al., U.S. Pat. No. 7,382,449; Nakamura, et al., U.S. Pat. No. 7,362,524; Tamada, U.S. Pat. No. 6,869,735).

Another method for applying the image forming material includes, for example “micro-contact printing”. The term “micro-contact printing” refers to the use of the relief patterns on a PDMS stamp to form patterns of self-assembled monolayers (SAMs) of an image-forming material on the surface of a thermoplastic material through conformal contact. Micro-contact printing differs from other printing methods, like inkjet printing or 3D printing, in the use of self-assembly (especially, the use of SAMs) to form micro patterns and microstructures of various image-forming materials. Such methods are well known in the art (Cracauer, et al., U.S. Pat. No. 6,981,445; Fujihira, et al., U.S. Pat. No. 6,868,786; Hall, et al., U.S. Pat. No. 6,792,856; Maracas, et al., U.S. Pat. No. 5,937,758).

“Soft-lithography” is intended to refer to a technique commonly known in the art. Soft-lithography uses a patterning device, such as a stamp, a mold or mask, having a transfer surface comprising a well defined pattern in conjunction with a receptive or conformable material to receive the transferred pattern. Microsized and nanosized structures are formed by material processing involving conformal contact on a molecular scale between the substrate and the transfer surface of the patterning device.

The term “receptive material” is intended to refer to a material which is capable of receiving a transferred pattern. In certain embodiments, the receptive material is a conformable material such as those typically used in soft lithography comprise of elastomeric materials, such as polydimethylsiloxane (PDMS), gelatin, agarose, polyethylene glycol, cellulose nitrate, polyacrylamide or chitosan. The thermoplastic receptive material, or thermoplastic material, is also a receptive material as it can be etched, for example.

“Imprint lithography” is intended to refer to a technique commonly known in the art. “Imprint lithography” typically refers to a three-dimensional patterning method which utilizes a patterning device, such as a stamp, a mold or mask.

A “mold” is intended to mean an imprint lithographic mold.

A “patterning device” is intended to be broadly interpreted as referring to a device that can be used to convey a patterned cross-section, corresponding to a pattern that is to be created in a target portion of the substrate.

A “pattern” is intended to mean a mark or design.

“Bonded” is intended to mean a fabrication process that joins materials, usually metals or thermoplastics, by causing coalescence. This is often done by melting the materials to form a pool of molten material that cools to become a strong joint, with pressure sometimes used in conjunction with heat, or by itself, to produce the bond.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

### METHODS FOR PREPARING WRINKLES AND ALIGNING CELLS

In one aspect, the present invention discloses a method for preparing an aligned cell population comprising the steps of 1) placing one or more cells on a surface having a texture, which texture has an average height of from about 100 nanometers to about 5 micrometers; and 2) allowing the cells to migrate or divide on the surface, thereby forming an aligned cell population on the textured surface.

In one aspect, the texture has an average height selected from the group consisting of about 200 nanometers, about 300 nanometers, about 500 nanometers, about 700 nanometers, about 1 micrometer, about 2 micrometers, about 3 micrometers, about 4 micrometers, and about 5 micrometers.

In one aspect, the texture has a periodicity in the range of from about 10 nanometers to about 600 nanometers. In another aspect, the texture has a periodicity selected from the group consisting of about 15 nanometers, about 30 nanometers, about 60 nanometers, and about 600 nanometers.

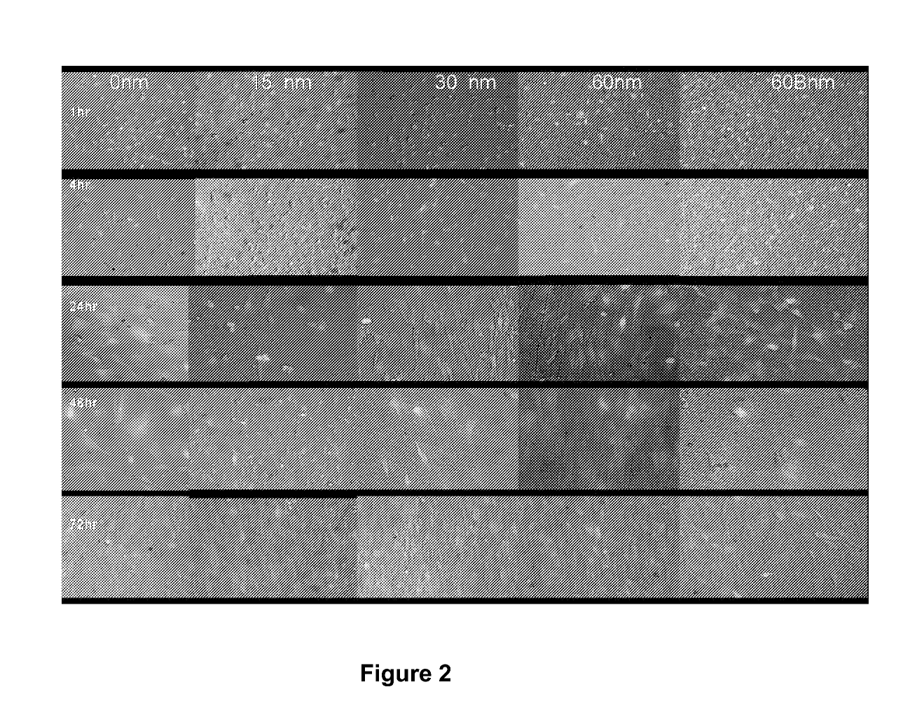

The optimal height and periodicity of the texture are cell type dependent. Referring to FIG. 2, the optimal height and periodicity of the texture for a cell type can be experimentally determined.

In one aspect, the textured surface is on a base made from a receptive material. Examples of receptive materials suitable for preparing the textured surface include, but are not limited to a material comprising a material of the group of polydimethylsiloxane, gelatin, agarose, polyethylene glycol, cellulose nitrate, polyacrylamide, or chitosan. In another aspect, the textured surface is on a base made of polydimethylsiloxane (PDMS).

A hydrophobic surface is known in the art to be beneficial for cell growth and alignment. Materials like PDMS have a hydrophobic surface and are therefore useful for preparing a textured surface of this invention. A hydrophilic surface, on the other hand, can help cell attachment. In one aspect of the invention, a temporary hydrophilic surface is created by applying an electric charge on the textured surface. The electric charge is removed after the cells attach to the surface.

In some embodiments, the preparation of the textured surface comprises the steps of: a) depositing a metal onto an unstressed or pre-stressed thermoplastic material; b) reducing the surface area of the receptive material by at least about 60%; and c) preparing the surface via lithography.

Steps a) and b) prepare a metal wrinkled surface on the unstressed or pre-stressed thermoplastic material. Methods for preparing the metal wrinkled surface can be found in PCT Patent Application No. PCT/US08/083,283, which is incorporated by reference in its entirety.

In some embodiment, the unstressed or pre-stressed thermoplastic material is a heat sensitive thermoplastic receptive material. In certain embodiments, the depositing of heat sensitive thermoplastic receptive material is by evaporating, which is a physical vapor deposition method to deposit a thin film of metal on the surface of a substrate. By heating a metal in a vacuum chamber to a hot enough temperature, the vapor pressure of the metal becomes significant and the metal evaporated. It recondenses on the target substrate. The height of the metal is dependent on length of processing time. The thermoplastic substrate must be far enough from the source such that the plastic does not heat up during deposition.

After the metal is deposited on the thermoplastic material, it is placed in an oven, or similar device, to be heated, and upon heating, because of the stiffness incompatibility between the metal and the shrinking thermoplastic, wrinkles form. The spacing between the metal wrinkles can be controlled by the amount of heating, and hence shrinkage.

Wrinkle height can be controlled by adjusting the metal film thickness. FIG. 17 of the PCT application PCT/US08/083,283 shows a plot of the maximum average wrinkle height as a function of metal layer thickness. Therefore, one can easily predict the spacing between and height of the metal wrinkles by adjusting the thickness of metal deposited onto the thermoplastic material and the time the thermoplastic material is heated. The thickness of metal deposited onto the thermoplastic material can be easily controlled using the metal deposition methods disclosed herein by adjusting parameters such as time, temperature, and the like. Such methods are well known to one of skill in the art.

Various heights can be achieved from about 2 nanometers to about 100 nanometers. In an particular embodiment, the height of the metal is about 2 nanometers. In an alternative embodiment, the height of the metal is about 5 nanometers, or alternatively, about 10 nanometers, or alternatively, about 20 nanometers, or alternatively, about 30 nanometers, or alternatively, about 40 nanometers, or alternatively, about 50 nanometers, or alternatively, about 60 nanometers, or alternatively, about 70 nanometers, or alternatively, about 80 nanometers, or alternatively, about 90 nanometers, or alternatively, about 100 nanometers.

In some embodiments, wrinkle heights can be achieved from about 100 nanometers to about 5 micrometers. In an particular embodiment, the height of the metal is about 200 nanometers. In an alternative embodiment, the height of the metal is about 200 nanometers, or alternatively, about 300 nanometers, or alternatively, about 500 nanometers, or alternatively, about 700 nanometers, or alternatively, about 1 micrometer, or alternatively, about 2 micrometers, or alternatively, about 3 micrometers, or alternatively, about 4 micrometers, or alternatively, less than about 5 micrometers.

In addition, the directionality of the wrinkles is controlled by grooving the substrate prior to metal deposition. Alternatively, the directionality of the wrinkles can be controlled by monodirectional shrinking using a uni-axially or bi-axially biasing thermoplastic receptive material. In one embodiment, the method to prepare a textured metal surface further comprises first heating a heat sensitive thermoplastic receptive material under conditions that reduce the size of the thermoplastic receptive material bi-axially by at least about 60%, followed by uni-axially biasing the thermoplastic receptive material to shrink along one axis or dimension prior to depositing a metal onto a heat sensitive thermoplastic receptive material, and reducing the material by at least about 60%, thereby preparing a textured metal surface.

In one aspect, the size of the textured metal surface is substantially the same as the thermoplastic receptive material before the receptive material was uni-axially biased. In one embodiment, the thermoplastic receptive material is uni-axially biased using heat.

It is contemplated that any metal can be deposited onto the thermoplastic receptive material to fabricate the metal wrinkles disclosed herein. In some embodiments, the metal is at least one of silver, gold or copper. Depending on the intended use of the metal surface, it may be desired that the metal be deposited in a given pattern or design. The metal can be deposited to only a desired area of the thermoplastic receptive material to form isolated metal sections or ‘islands’ on the thermoplastic receptive material. Methods for the controlled deposition of metals are well known in the art.

The periodicity of the wrinkle as the wavelength of the wrinkles scale according to the thickness to the ¾th power. Therefore, tighter wrinkles are achieved by changing the thickness, or height of the metal layer.

It is contemplated that any thermoplastic material can be used in the methods disclosed herein. In one aspect of the disclosed invention, the thermoplastic materials 5 are those which shrink uniformly without substantial distortion. Suitable thermoplastic materials 5 for inclusion in the methods of this invention include, for example, high molecular weight polymers such as acrylonitrile butadiene styrene (ABS), acrylic, celluloid, cellulose acetate, ethylene-vinyl acetate (EVA), ethylene vinyl alcohol (EVAL), fluoroplastics (PTFEs, including FEP, PFA, CTFE, ECTFE, ETFE), ionomers kydex, a trademarked acrylic/PVC alloy, liquid crystal polymer (LCP), polyacetal (POM or Acetal), polyacrylates (Acrylic), polyacrylonitrile (PAN or Acrylonitrile), polyamide (PA or Nylon), polyamide-imide (PAI), polyaryletherketone (PAEK or Ketone), polybutadiene (PBD), polybutylene (PB), polybutylene terephthalate (PBT), polyethylene terephthalate (PET), Polycyclohexylene Dimethylene Terephthalate (PCT), polycarbonate (PC), polyhydroxyalkanoates (PHAs), polyketone (PK), polyester polyethylene (PE), polyetheretherketone (PEEK), polyetherimide (PEI), polyethersulfone (PES), polysulfone polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC) and spectralon. In one embodiment, the thermoplastic material is polystyrene. In one embodiment, the thermoplastic material 5 is polystyrene.

Alternative embodiments of the above-noted methods include, but are not limited to the application of heat to reduce the size of the thermoplastic receptive material 5 by at least 65%, or alternatively, at least 70%, or alternatively, at least 75%, or alternatively, at least 80%, or alternatively, at least 85%, or alternatively, at least 90%.

Thus in some embodiments, the pre-stressed thermoplastic material is bi-axially biased. In some embodiments, the pre-stressed thermoplastic material is uni-axially biased. In other embodiments it is not pre-stressed, i.e., unstressed.

In some embodiments, the metal is deposited by sputter coating, evaporation or chemical vapor deposition.

In some embodiments, the unstressed or pre-stressed thermoplastic material is reduced to achieve a surface texture having a periodicity in the range of from about 10 nanometers to about 5 micrometers.

In some embodiments, the unstressed or pre-stressed thermoplastic material is reduced to achieve a surface texture having a periodicity in the range of from about 10 nanometers to about 600 nanometers. In one aspect, the unstressed or pre-stressed thermoplastic material is reduced to achieve a surface texture having a periodicity in the range of from about 15 nanometers to about 100 nanometers. In yet another aspect, the unstressed or pre-stressed thermoplastic material is reduced to achieve a surface texture having a periodicity selected from the group consisting of about 15 nanometers, about 30 nanometers, about 60 nanometers, and about 600 nanometers.

In some embodiments, the metal is deposited in a desired pattern.

In some embodiments, the heat sensitive thermoplastic material is reduced by heating. In some embodiments, the temperature used to heat and reduce the size of the thermoplastic material is from about 100° C. to about 250° C., or alternatively from about 120° C. to about 220° C., or alternatively from about 150° C. to about 200°, or alternatively from about 180° C. to about 190° C., or alternatively about 185° C.

After the thermoplastic material has been reduced in size to create a mold.

Soft or imprint lithography is used to create the receptive material. The molding or the lithography comprises, or alternatively consists essentially of, or yet further consists of a process such as soft lithography or imprint lithography. Examples of receptive materials include without limitation a material comprising one or more of polydimethylsiloxane, gelatin, agrose, polyethylene glycol, cellulose nitrate, polyacrylamide, and chitosan.

In on aspect, the lithography uses a thermoplastic material.

In one aspect, the material used in lithography, such as PDMS, is poured onto the textured metal surface, which serves as the mold, as in typical soft lithography, and cured at 110° Celsius for 10 minutes. The cured PDMS device is then peeled off the mold and bonded using a hand-held corona discharger (Haubert K., et al. (2006) Lab Chip Technical Note 6: 1548-1549). The whole process from device design conception to working device can be completed within minutes.

In some embodiments, to induce the formation of cell growth or alignment, the textured surface of the material, such as PDMS, can be soaked in polar and non-polar solvents such as pentane for 12 hours, followed by a solvent change where new pentane is added and is further soaked for 12 hours, next the pentane solvent is replaced with xylene for 7 hours and is replaced with new xylene for another 12 hours, last the microwells are soaked in ethanol for 12 hours prior to use. To simplify the protocol and save time, the first solvent is generally used to swell PDMS as much as possible, then followed by de-swelling gradually. Solvents that swelled PDMS the least: water, nitromethane, dimethyl sulfoxide, ethylene glycol, perfluorotributylamine, perfluorodecalin, acetonitrile, and propylene carbonate. Solvents that swelled PDMS the most: diisopropylamine, triethylamine, pentane, and xylenes. For an example of swelling and de-swelling procedure, soak in pentane for 24 hours; pentane 7 hours; then xylene isomers plus ethylbenzene 98.5% 1-2 hours; then xylenes for 16 hours; xylenes for 7 hours; then EtOH 1-2 hours, then EtOH again for 16 hours, and finally EtOH for 7 hours. Then soak in about 1 L of sterile DI water overnight and dry at 70° C. overnight.

Various types of cells may be grown or aligned on the textured surface of the present invention. In one aspect, the cell is an isolated prokaryotic or eukaryotic cell. In another aspect, the cell is an isolated eukaryotic cell.

In one aspect, the isolated eukaryotic cell may be an isolated stem cell. In some embodiments, the isolated stem cell is selected from the group consisting of an embryonic stem cell, a pluriopotent stem cell, a somatic stem cell and an induced pluripotent stem cell (iPS stem cell). Methods to grow and culture such cells are known in the art. See for example, US Patent Publ. Nos. 2009/0081784; 2009/0075374; 2009/0068742; and 2009/0047263.

In one aspect, the isolated stem cell is of animal origin, i.e., an animal stem cell. In some embodiments, the animal origin is mammalian, simian, bovine or murine. In one aspect, the animal origin is human.

In another aspect, the isolated eukaryotic cell is a fetal or neonatal cell.

In one aspect, the eukaryotic cell is selected from the group consisting of a smooth muscle cell, a bladder smooth muscle cell, a keratocyte, a corneal epithelial cell, an endothelial cell, a vascular endothelial cell, an osteoblast cell, a fibroblast cell, a myoblast cell, a nerve cell, a skin cell, and a cardiac cell. In another aspect, the eukaryotic cell is a fetal or neonatal cardiac cell. As is apparent to the skilled artisan, after culturing the feeder cells on the surface of the invention, they can be isolated or removed from the surface for further in vitro of in vivo use. Aligned cell cultures or cell populations prepared by the methods of this invention are further provided herein.

The present invention can be used to form a cardiac patch by aligning cardiac cells on the textured surface. Cardiac patches can be generated with methods known in the art, for example, Stevens et al. discloses a method to grow a human cardiac patch from embryonic stem cells (Stevens et al. (2009) Tissue Engineering 15:DOI: 10.1089/ten.tea.2008.0151). Further provided by this invention is a cardiac patch formed by the method of this invention.

Thus in one aspect, fetal or neonatal heart cells of are allowed to form a cardiac patch.

Also provided is a method for assaying a potential agent for the ability to affect cell migration, growth and/or differentiation of an isolated stem cell or other cell type comprising the steps of placing a cell with an agent on a surface of a material of this invention and allowing the cell to migrate or divide on the wrinkled or textured surface and assaying for the agent's effect on the cell's migration, growth and/or differentiation, wherein the surface has a texture having an average height of from about 100 nanometers to about 5 micrometers.

Also provided is a kit for use in preparing an aligned cell population comprising a textured material having a surface as described above and instructions to prepare an aligned cell population, which surface has a texture that has an average height of from about 100 nanometers to about 5 micrometers.

### EXAMPLES

The present technology is further understood by reference to the following examples. The present technology is not limited in scope by the examples, which are intended as illustrations of aspects of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

### Example 1

### Rapid Generation of Textured Surface for Cell Alignment

A piece of unshrunken polystyrene sheet (PS) is cut (approximately 2.5×3 cms). It is cleaned with isopropanol and distilled water and allowed to dry. A 60 nm layer of silver is deposited on the PS using a metal sputter. Alternatively, metals can be deposited onto the shrinkable thermoplastic by either thermal evaporation or sputtering. The metal coated PS is fixed by the opposite side to a glass slide using binder clips and baked for 10 minutes at 165° C. This will shrink the PS sheet in one direction creating aligned metal wrinkles. The thickness, or height of the deposited metal is dependent on length of processing time. The plastic substrate should be far enough from the source such that the plastic does not heat up during deposition. A wide range of thicknesses, or heights, of deposited metal are accomplished, from about 5 nanometers to about 90 nanometers.

Upon heating, because of the stiffness incompatibility between the metal and the shrinking thermoplastic, wrinkles (textures) formed. The spacing between the wrinkles can be controlled by the amount of heating, and hence shrinkage. In addition, the directionality of the wrinkles can be controlled by grooving the substrate prior to metal deposition. Finally, the periodicity of the wrinkle as the wavelength of the wrinkles scale according to the thickness to the ¾th power. Therefore, tighter wrinkles were achieved by changing the thickness, or height of the metal layer. After heating, the PS is cooled to 75° C. to avoid cracking. The wrinkled mold is then attached to a petri dish with double-sided tape.

Then PDMS is poured onto the mold as in typical soft lithography, and cured at 110° Celsius for 10 minutes. The cured PDMS device is then peeled off the mold and bonded using a hand-held corona discharger (Haubert K., et al. (2006) Lab Chip Technical Note 6: 1548-1549). The whole process from device design conception to working device can be completed within minutes.

Alternatively, PDMS (1:10) is added to the mold and allowed to polymerize at 75° C. for 30 to 45 minutes. A 0.5 by 0.5 cm² piece of micropatterned PDMS is cut and cleaned using a piece of tape to remove any dirt or undesired materials. The patterned PDMS is bonded to a piece of glass with uncured PDMS (1:10) and left to polymerize at 75° C. for 30 to 40 minutes. The PDMS is then treated with a corona discharger for 15 seconds and subsequently sterilized with 70% ethanol solution for 15 minutes under an ultraviolet light source. The chip is then washed twice with sterile phosphate buffered saline (PBS) and placed on a 48 well-plate.

### Example 2

### Aligning Cardiac Cells on Textured Surface

Prior to placing cells onto the textured surface of PDMS. The surface was treated with solvent as described previously. Prior to loading the cells, the PDMS device was electrically charged to create a temporary hydrophilic environment facilitating cell attachments. Alternatively, the substrate can be coated with laminin, fibronectin or collagen IV, depending on the cell type. For example, for cardiomyoctyes, laminin and fibronectin were used at a concentration of 1 μg/cm². For stromal cells, fibronectin was used at a concentration of 0.1 μg/cm². The coated substrates were allowed to air-dry in a biohood overnight.

The dried, coated substrate were rinsed with sterile PBS. 500 μl of media was added to each well. Cells should be seeded at a high density (for example, for cardiomyocytes, use 0.3×10⁵ cells/cm²). The cells are incubated in 95% air/5% CO2 at 37° C. for one (1) hour. Media is changed every 72 hours or at any other time depending on cell type.

The PDMS device was placed in a well, and fetal cardiac cells isolated from mice were placed on the PDMS device in the well. The wells were examined under microscope after 4 hours, 24 hours, 48 hours, and 72 hours to examine alignment of the cardiac cell.

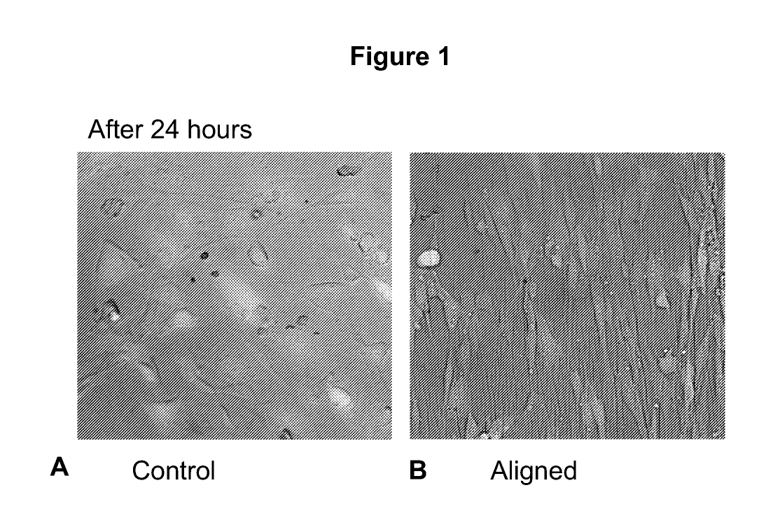

As shown in FIG. 1, cells did not migrate or migrated at a random direction on a flat surface. On the textured PDMS surface, the cells migrated and aligned in the direction of the texture. As shown in FIG. 2, cardiac patches started to form at about 24 hours. The alignment also depended on periodicity of the texture and time of culturing.

While the present invention is exemplified and illustrated by the use of polystyrene sheets to fabricate channel structures and molds, it would be obvious to those of skill in the art that any thermoplastic receptive material that can be patterned to control the dimensions of the channel defining walls and thereby their size, can be used to fabricate the devices disclosed and claimed herein. In addition, although several other embodiments of the invention are described herein in detail, it will be understood by those skilled in the art that variations may be made thereto without departing from the spirit of the invention or the scope of the appended claims.

What is claimed is:

1. A method for preparing an aligned cell population comprising culturing one or more isolated cells on a surface having a texture, which texture has an average height of from about 100 nanometers to about 5 micrometers, thereby forming an aligned cell population on the surface having a texture.

2. The method of claim 1, wherein the texture has an average height selected from the group consisting of about 200 nanometers, about 300 nanometers, about 500 nanometers, about 700 nanometers, about 1 micrometer, about 2 micrometers, about 3 micrometers, about 4 micrometers, and about 5 micrometers.

3. The method of claim 1 or claim 2, wherein the texture has a periodicity in the range of from about 10 nanometers to about 600 nanometers.

4. The method of any of claims 1 to 3, wherein the texture has a periodicity selected from the group consisting of about 15 nanometers, about 30 nanometers, about 60 nanometers, and about 600 nanometers.

5. The method of any of claims 1 to 4, wherein the textured surface is on a material comprising polydimethylsiloxane, gelatin, agrose, polyethylene glycol, cellulose nitrate, polyacrylamide or chitosan.

6. The method of any of claims 1 to 4, wherein the textured surface is on polydimethylsiloxane.

7. The method of any of claims 1 to 6, further comprising isolating the cells from the textured surface.

8. The method of any of claims 1 to 7, wherein the isolated one or more cells is prokaryotic cell or a eukaryotic cell.

9. The method of any of claims 1 to 7, wherein the isolated one or more cells is a stem cell.

10. An isolated population of substantially aligned cells prepared by the method of any of claims 1 to 9.

11. The method of any of claims 1 to 9, wherein the surface having the texture is prepared by a method comprising:
a) depositing a metal onto a unstressed or pre-stressed thermoplastic material;
b) reducing the surface area of the thermoplastic material by at least about 60%; and
c) preparing the surface via lithography.

12. The method of claim 11, wherein the pre-stressed thermoplastic material is uni-axially biased or bi-axially biased.

13. The method of any of claim 11 or 12, wherein the metal is deposited by sputter coating, evaporation or chemical vapor deposition.

14. The method of any of claims 11 to 13, wherein the metal is deposited in a thickness of from about 2 nanometers to about 100 nanometers.

15. The method of any of claims 11 to 14, wherein the thermoplastic material is reduced to achieve a surface texture having a periodicity in the range of from about 10 nanometers to about 5 micrometers.

16. The method of any of claims 11 to 14, wherein the pre-stressed thermoplastic material is reduced to achieve a surface texture having a periodicity in the range of from about 10 nanometers to about 600 nanometers.

17. The method of any of claims 11 to 14, wherein the pre-stressed thermoplastic material is reduced to achieve a surface texture having a periodicity in the range of from about 15 nanometers to about 100 nanometers.

18. The method of any of claims 11 to 14, wherein the pre-stressed thermoplastic material is reduced to achieve a surface texture having a periodicity selected from the group consisting of about 15 nanometers, about 30 nanometers, about 60 nanometers, and about 600 nanometers.

19. The method of any of claims 11 to 18, wherein the metal is deposited in a desired pattern on the unstressed or pre-stressed receptive thermoplastic material.

20. The method of any of claims 11 to 19, wherein the unstressed or pre-stressed thermoplastic material is reduced by heating.

21. The method of any of claims 11 to 20, wherein the lithography of step c) comprises soft lithography or imprint lithography.

22. The method of any of claims 11 to 21, wherein the surface having the texture comprises a material of the group of polydimethylsiloxane, gelatin, agrose, polyethylene glycol, cellulose nitrate, polyacrylamide and chitosan.

23. The method of claim 9, wherein the isolated stem cell is selected from the group consisting of an embryonic stem cell, a pluriopotent stem cell, a somatic stem cell and an iPS stem cell.

24. The method of claim 22, wherein the isolated stem cell is an animal stem cell.

25. The method of claim 24, wherein the animal stem cell is mammalian, simian, bovine or murine.

26. The method of claim 25, wherein the animal stem cell is a human stem cell.

27. The method of claim 21, wherein the isolated eukaryotic cell is a fetal or neonatal cell.

28. The method of any of claims 21 to 27, wherein the eukaryotic cell is selected from the group consisting of a smooth muscle cell, a bladder smooth muscle cell, a keratocyte, a corneal epithelial cell, an endothelial cell, a vascular endothelial cell, an osteoblast cell, a fibroblast cell, a myoblast cell, a nerve cell, a skin cell, and a cardiac cell.

29. The method of claim 28, wherein the eukaryotic cell is a fetal or neonatal cardiac cell.

30. The method of claim 28, wherein the fetal or neonatal heart cell is cultured under conditions to form a cardiac patch.

31. A method for assaying a potential agent for the ability to affect cell migration, growth and/or differentiation of cell, comprising the steps of placing a cell with an agent on a surface of a surface of any of claims 1 to 10 and allowing the cell to migrate or divide on surface and assaying the agent's ability to affect cell migration, growth and/or differentiation of the cell.

32. A kit for use in preparing an aligned cell population comprising a surface of any of claims 1 to 10 and instructions for use.
